(19) United States
(12) Patent Application Publication
(10) Pub. No.: US 2009/0263429 A1
(43) Pub. Date: Oct. 22, 2009

(54) Method of Preparing a Hydrogel

(75) Inventors: Rein Vincent Ulijn, St. Helens (GB); Sophie Toledano, Paris (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: The University of Manchester, Manchester (GB)

(21) Appl. No.: 11/991,768

(22) PCT Filed: Sep. 7, 2006
(86) PCT No.: PCT/GB2006/003325

§ 371 Date: Mar. 7, 2008

(30) Foreign Application Priority Data
Sep. 7, 2005 (GB) 0518220.9

(51) Int. Cl. A61L 15/60; C12P 13/02; C12P 7/62; C12P 21/06; A61K 38/46; A61P 17/02; C12N 5/00

(52) U.S. Cl. 424/400; 435/129; 435/135; 435/68.1; 424/94.6; 435/404; 424/93.7

(57) ABSTRACT

A method of preparing a hydrogel comprises: (i) reacting a first molecule comprising a carboxylic acid group and second molecule comprising an amine group or an alcohol group with a hydrolase enzyme to form a product comprising an amide bond or ester bond, wherein the hydrolase enzyme would normally catalyse the production of an amine or an alcohol from an amide or an ester under physiological conditions; and (ii) maintaining the product comprising an amide or ester bond under conditions suitable to allow hydrogel formation.

The present invention relates to methods of preparing a hydrogel, and in particular, to methods of synthesizing hydrogels made up of self-assembling molecules. The invention extends to hydrogels prepared using the method, and to uses thereof, for example in medicine.

The self-assembly of macroscopic materials from small molecule building blocks, provides a powerful route for designing molecular biomaterials, which can be used in a range of biological applications. Such materials may be composed of macromolecules such as, proteins or lipids etc. The ability to control the assembly of these structures on demand by the application of an external stimulus is of particular value, especially in biomedical contexts. For example, in minimal invasive surgery for tissue repair, a liquid precursor molecule is mixed with cells, injected in the body to form a gel scaffold in situ for tissue re-growth. Gelation-on-demand is also of use for in vitro studies, where 3D hydrogel scaffolds are increasingly used as suitable “wet” environments to study cell behaviour.

Stimuli that have been used to cause gelation of the liquid precursor molecule include a variety of chemical and physical means, including changes in ionic strength, pH, temperature and addition of certain chemical entities. However, a problem with using these stimuli in biomedical applications is that they can be non-selective, and can cause other unwanted effects. A further problem is that biological molecules tend to be sensitive to ionic strength, pH and temperature, and hence, varying any of these parameters will tend to disrupt biological interactions, and in some cases destroy the biological molecules themselves. Accordingly, medical practitioners tend to avoid using such stimuli. There is therefore a need to provide improved ways in which gelation of liquid precursors can be triggered.

Another form of stimulus that has been investigated for use in the transformation of a liquid precursor into a gel is the use of enzymes. Enzymes are biological catalysts and therefore it was hoped that the use of enzymes would be less disruptive and more selective in biomedical settings. Previous work in the area of enzyme-assisted assembly includes the use of protein cross-linking enzymes, such as transglutaminase (TGase) to trigger assembly of peptide conjugates. The use of phosphorylation and dephosphorylation to control beta sheet assembly by a kinase and an alkaline phosphatase has also been demonstrated. Other research groups have reported the controlled self-assembly by enzyme-triggered intramolecular acyl migration in modified peptides.

However, there are a number of problems associated with each of these enzymatic systems. Firstly, they often consist of non-natural building blocks, and are therefore not ultimately compatible with biomedical systems. Secondly, these systems often involve hydrolysis of precursor molecules, thus releasing stoichiometric amounts of hydrolysis products into the immediate environment. Thirdly, the enzymes that are used to trigger self-assembly may not be naturally present in biomedical contexts, and would need to be added for self-assembly in situ.

It is therefore an object of the present invention to obviate or mitigate one or more of the problems of the prior art, whether identified herein or elsewhere, and to provide an improved method for preparing a hydrogel.

According to a first aspect of the present invention, there is provided a method of preparing a hydrogel, the method comprising:—

(i) reacting a first molecule comprising a carboxylic acid and second molecule comprising an amine or an alcohol with a hydrolase enzyme to form a product comprising an amide bond or ester bond, wherein the hydrolase enzyme would normally catalyse the production of an amine or an alcohol from an amide or an ester under physiological conditions; and

(ii) maintaining the product comprising an amide or ester bond under conditions suitable to allow hydrogel formation.

According to a second aspect of the invention, there is provided a hydrogel prepared using a method according to the first aspect.

The inventors have demonstrated for the first time a method for selectively triggering the synthesis and subsequent self-assembly of amide molecules or ester molecules into hydrogel materials. This is achievable by exploiting as a biological stimulus, the reverse catalytic action of a hydrolase enzyme. More specifically, the method comprises stimulating gelation of otherwise non-gelling precursor molecules (ie. the first molecule comprising a carboxylic acid and second molecule comprising an amine or alcohol) to form a hydrogel by using hydrolase enzymes that have evolved to hydrolyse peptide bonds under normal physiological conditions, to perform the reverse reaction (i.e. peptide synthesis or reversed hydrolysis). Accordingly, the inventors believe that the method according to the invention is completely unexpected, as it makes use of hydrolase enzymes in a non-obvious manner by harnessing them to synthesise amides or esters, whereas they would normally carry out the opposite reaction and actively hydrolyse amides or esters in aqueous media under normal physiological conditions.

While the inventors do not wish to be bound by any hypothesis, they believe that the formation of the hydrogel in step (ii) of the method results in the ‘net’ removal of the product formed by the reaction catalysed by the hydrolase enzyme in step (i) of the method. The inventors believe that this removal of the product, be it either an amide or an ester, causes a shift in the equilibrium of the reaction such that the hydrolase is induced to carry out reverse hydrolysis. Hence, the inventors believe that step (ii) of the method is effectively driving step (i). The skilled technician would not expect that a hydrolase enzyme could be used in a reverse hydrolysis reaction to form a hydrogel. Hence, the inventors maintain that this is use of a hydrolase enzyme is counter-intuitive to the normal action of a hydrolase.

Furthermore, this enzymatic approach has a number of significant advantages over currently used chemical and physical means, which include changes in ionic strength, pH, temperature and addition of certain chemical entities. Firstly, enzymes are uniquely chemo-, regio-, and enantioselective. Secondly, enzymes naturally work under mild conditions (aqueous, pH 5-8). Thirdly, a number of enzymes play key roles as selective catalysts in cell pathways and disease states, and so their function may be harnessed in a range of medical applications.

By the term “hydrogel”, we mean a gel in which water is the major dispersion medium. Hence, preferably, components or subunits of the hydrogel, i.e. the self-assembled amides or esters, are dispersed within water. Preferably, the hydrogel comprises at least 80% (w/w) water, more preferably, at least 85% (w/w) water, and more preferably, at least 90% (w/w), even more preferably, at least 95% (w/w) water.

The self-assembling subunits of the hydrogel (i.e. the amides or esters) may have a molecular weight of between 100 and 30,000 Da, more preferably, between 200 and 20,000 Da, even preferably, between 300 and 15,000 Da, and most preferably, between 500 and 10,000 Da.

By the term “hydrolase”, we mean an enzyme that is adapted under normal physiological conditions to break a chemical bond by hydrolysis. It will be appreciated that hydrolases are classified as EC3 in the EC number classification index. For example, the hydrolase may be a nuclease, glycosylase, esterase, or protease. Esterases and proteases use water for the catalytic reaction, and are therefore classified as hydrolases.

By the term “esterase”, we mean an enzyme that catalyzes the hydrolysis of esters (—COO—). It will be appreciated that esters are normally formed by the reaction between a carboxylic acid and an alcohol.

By the term “protease”, we mean an enzyme that digests or breaks a peptide bond (—CONH—) of a peptide, and the process is called proteolytic cleavage. It will be appreciated that peptides are normally formed by the reaction between a carboxylic acid and an amine. Examples of suitable protease will be known to the skilled technician, for example, thermolysin or chymotrypsin.

The inventors believe that the method according to the invention will have considerable utility for producing a hydrogel on demand, which would be particularly useful in many biomedical settings. The specific utility of the hydrogel will depend on whether it comprises a plurality of self-assembled esters or self-assembled amides.

In some biomedical applications, the hydrogel may comprise a plurality of esters. It will be appreciated that there are many types of biologically useful compounds, which comprise ester bonds. For example, suitable ester compounds may include fats, fatty acids, lipids, which may be used to form glycolipids and phospholipids (e.g. phosphatidyl choline).

One example of a preferred ester, which may be formed in step (i) of the method is an acylaglyceride, the structure of which will be known to the skilled technician. An acylglyceride may be prepared by reacting a fatty acid molecule (i.e. the first molecule comprising a carboxylic acid group) and glycerol (ie. the second molecule comprising an alcohol group).

Hence, in one embodiment of the method, the alcohol used may be a primary, secondary, or tertiary alcohol, such that upon reaction with the carboxylic acid in step (i), an ester is formed.

The relevant reaction scheme may be shown as reaction scheme 1 as follows:—

R¹—COOH+HO—R²→H₂O+R¹—COO—R²

wherein, R¹, and R² may be independently selected from a group consisting of a hydrogen; a linear alkyl group; a branched alkyl group; and a side chain group of an amino acid residue. It will be appreciated that R¹—COOH is the first molecule, and HO—R² is the second molecule used in the reaction according to the invention.

Suitable alkyl groups may comprise a C₁-C₂₀ chain, and preferably, a C₁-C₁₅ chain. It is envisaged that the alkyl group may comprise a C₁-C₁₀ chain, and more preferably, a C₁-C₆ chain, and most preferably a C₁-C₃ chain. The chain may be straight or branched. However, preferably, the chain is straight. The alkyl group or alkyl chain may be a methyl, ethyl, propyl, butyl, or a pentyl chain.

R¹, and R² may independently comprise a side chain group of an amino acid residue. It is especially preferred that R¹, and R² comprise an amino acid side chain group of a DNA encoded amino acid. The amino acid side chain group may be independently selected from the repertoire of non-naturally occurring and 20 naturally occurring amino acids. Hence, R¹, and R² may comprise an amino acid side chain group of an acidic, basic, hydrophobic or a hydrophilic amino acid residue.

Hence, in this embodiment of the invention, by the term “physiological conditions”, we mean that the normal thermodynamics of the reaction catalysed by the hydrolase enzyme would drive the hydrolysis of the ester, R¹—COO—R², (i.e. from right to left of reaction scheme 1) to produce a carboxylic acid and an alcohol shown on the left of the arrow in reaction scheme 1. However, surprisingly, the inventors have found that the method according to the first aspect incorporating the action of the hydrolase enzyme may be used to drive the reverse reaction (i.e. from left to right of reaction scheme 1), thereby producing the ester, R¹—COO—R², shown on the right of the arrow in reaction scheme 2. As discussed herein, while the inventors do not wish to be bound by any hypothesis, they believe that the formation of the hydrogel in step (ii) of the method causes the removal of the ester product molecules, which in turn causes a shift in the reaction equilibrium such that the hydrolase causes synthesis of further ester product. Hence, the inventors believe that step (ii) of the method is effectively driving step (i). The inventors believe that this shifting of the thermodynamics of the reaction scheme 1 by the formation of self-assembling hydrogel could not have been predicted.

Accordingly, the product comprising an ester bond formed in step (i) of the method may comprise a fat, fatty acid or lipid. The skilled technician will appreciate that fats or lipids comprise ester bonds, and may therefore be defined as a product comprising an ester bond as in the method according to the first aspect.

Hence, the hydrolase preferably catalyses the reaction between the first molecule comprising the carboxylic acid and the second molecule comprising the alcohol group to thereby form the ester product. The esters formed as a result of this reaction are then adapted to self-assemble to form the hydrogel in step (ii) of the method.

In other biomedical applications, it is preferred that the hydrogel may comprise a plurality of peptides. The second molecule comprising the amine used in the method may be a primary, secondary, or tertiary amine, or amino acid, such that upon reaction with the carboxylic acid in step (i), a peptide is formed.

The relevant reaction scheme may be shown as reaction scheme 2 as follows:—

R¹—COOH+H₂N—R²→H₂O+R¹—CONH—R²

wherein, R¹, and R² may be independently selected from a group consisting of a hydrogen; a linear alkyl group; a branched alkyl group; and a side chain group of an amino acid residue. It will be appreciated that R¹—COOH is the first molecule, and H₂N—R² is the second molecule used in the reaction according to the invention.

Suitable alkyl groups may comprise a C₁-C₂₀ chain, and preferably, a C₁-C₁₅ chain. It is envisaged that the alkyl group may comprise a C₁-C₁₀ chain, and more preferably, a C₁-C₆ chain, and most preferably a C₁-C₃ chain. The chain may be straight or branched. However, preferably, the chain is straight. The alkyl group or alkyl chain may be a methyl, ethyl, propyl, butyl, or a pentyl chain.

R¹, and R² may comprise a side chain group of an amino acid residue. It is therefore preferred that R¹, and R¹ is either an amino acid, or may comprise an amino acid side chain group of a DNA encoded amino acid. The amino acid side chain group may be independently selected from the repertoire of non-naturally occurring and 20 naturally occurring amino acids. Hence, R¹, and R² may comprise an amino acid side chain group of an acidic, basic, hydrophobic or a hydrophilic amino acid residue.

Hence, by the term “physiological conditions”, we mean that the normal thermodynamics of the reaction catalysed by the hydrolase enzyme would drive the hydrolysis of the amide, R¹—CONH—R², (i.e. from right to left of reaction scheme 2) to produce a carboxylic acid and an amine shown on the left of the arrow in reaction scheme 2. However, surprisingly, the inventors have found that the method according to the first aspect may be used to drive the reverse reaction (i.e. from left to right of reaction scheme 1), thereby producing the amide R¹—CONH—R² shown on the right of the arrow in reaction scheme 1. By the term “amide”, we mean an organic compound containing the group R¹—CONH—R². As discussed herein, while the inventors do not wish to be bound by any hypothesis, they believe that the formation of the hydrogel in step (ii) of the method causes the removal of the amide product molecules, which in turn causes a shift in the reaction equilibrium such that the hydrolase causes synthesis of further amide product. Hence, the inventors believe that step (ii) of the method is effectively driving step (i). The inventors believe that this shifting of the thermodynamics of the reaction scheme 1 by the formation of self-assembling hydrogel could not have been predicted.

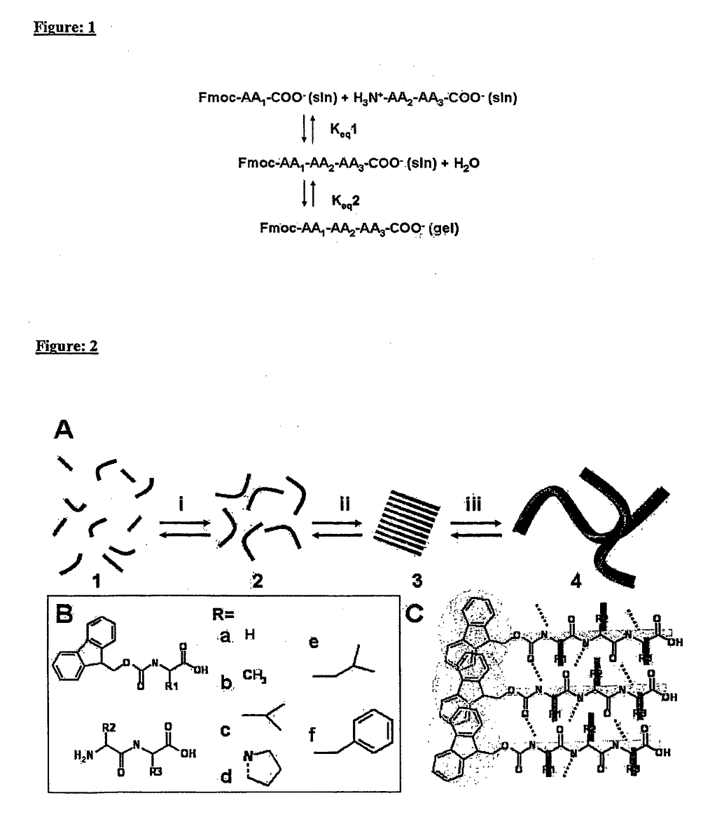

Accordingly, the product comprising an amide bond formed in step (i) of the method may comprise a peptide, polypeptide or protein. FIGS. 1 and 2 illustrate the reactions involved. The skilled technician will appreciate that peptides comprise amide bonds, and may therefore be defined as a product comprising an amide bond as in the method according to the first aspect.

Each peptide formed in step (i) of the method preferably comprises at least two amino acid residues, and preferably, at least three amino acid residues. Hence, in embodiments where the method according to the invention is used to prepare a hydrogel comprising a plurality of peptide molecules, the first molecule comprising a carboxylic acid used in step (i) comprises at least one amino acid residue, and more preferably, a plurality of amino acid residues.

Furthermore, in embodiments where the method according to the invention is used to prepare a peptide, the second molecule comprising the amine used in step (i) preferably comprises at least one amino acid residue, and more preferably, a plurality of amino acid residues.

Hence, in preferred embodiments of the invention, the hydrolase catalyses the reaction between two or more amino acids. Preferably, a carboxylic group on one amino acid reacts with a amine group on another amino acid to thereby form a peptide product. Accordingly, different numbers of amino acids may be reacted together to form peptides of various lengths. For example, step (i) may comprise reacting an amino acid with a dipeptide to form a tripeptide, or reacting a dipeptide with another dipeptide to form a tetrapeptide, and so on. The peptides formed as a result of this reaction are then adapted to self-assemble to form the hydrogel in step (ii) of the method.

Hence, the preferred reaction scheme may be shown as reaction scheme 3 as follows: —

AAₙ+AAₘ→H₂O+AAₙ₊ₘ

wherein AA represents an amino acid, n represents the number of amino acids on the first molecule, and m represents the number of amino acids on the second molecule (n or m may be 1-500, preferably 1-250). Hence, it will be appreciated that peptides may be prepared in step (i) having various numbers of amino acid residues.

Accordingly, the method according to the invention preferably comprises preparing a hydrogel, which comprises a plurality of self-assembling peptides, in which step (i) preferably comprises reacting at least two amino acid residues with a hydrolase enzyme to form a peptide, wherein the hydrolase enzyme would normally catalyse the production of amino acids from the peptide under physiological conditions; and wherein in step (ii) comprises allowing the peptide molecules formed in step (i) to self-assemble with each other to thereby form the hydrogel.

The inventors believe that using enzymes to trigger the self-assembly of peptides in the two-step method is attractive since hydrolases have a high specificity for the peptides they hydrolyse/synthesise, thus allowing for peptide hydrogels to be programmed to respond to certain hydrolase enzymes only. While the inventors do not wish to be bound by any hypothesis, they believe that self-assembly of the peptides in step (ii) of the method provides a means of thermodynamically stabilizing the peptides into a hydrogel structure, which the inventors believe may act as a driving force to favour peptide synthesis in step (i).

Preferably, the reaction in step (i) of the method is carried out in aqueous medium. Hence, by the term “aqueous media”, we mean a solution, which comprises water.

The amide or ester formed in step (i) of the method are adapted to self-assemble with each other to produce the hydrogel. The inventors hypothesise that self-assembly of these hydrogel components can occur via various mechanisms, such as by means of ionic interactions, hydrophobic interactions, hydrogen bonding, or by Van der Waals forces.

However, it preferred that the hydrogel components are adapted to assemble with each other due to the presence of an aromatic stacking ligand in the or each amino acid reacted in step (i).

Preferably, the carboxylic acid, or the amine or the alcohol in step (i) of the method comprises an aromatic stacking ligand.

By the term “aromatic stacking ligand”, we mean an aromatic molecule comprising at least one benzene ring, or a related planar, cyclic structure with a delocalised electron structure, such as pyridine, furan or thiophene. More generally, the aromatic ligand may be covalently attached either to the N or C terminus or side chain of an amino acid and, preferably adhere to the 4n+2 (Huckel) rule. It is preferred that the stacking ligand is adapted to interact with at least one other aromatic stacking ligand. Hence, the molecules are able to self-assemble with each other. Surprisingly, such self-assembly of the stacking ligands results in the self-assembly of the amides (e.g peptides) or esters (e.g. fats) to which they are attached. As the amides or esters assemble together, the hydrogel is formed under biologically acceptable conditions.

Examples of a suitable aromatic stacking ligand, which may be attached to the amide or ester in the hydrogel include any aromatic compound, which comprises at least one benzene ring. The skilled technician will appreciate that there are many different types of aromatic compounds available that could be attached to the amide or ester in the hydrogel, and which would interact with each other to form a hydrogel. However, examples of suitable aromatic stacking ligand to which the amids or ester may be attached include benzoyl (Bz) or carboxybenzoyl (Cbz), both of which are common protecting groups used in peptide synthesis, and which will be known to the skilled technician.

However, a preferred aromatic stacking ligand comprises Fmoc (fluorenylmethoxycarbonyl), which is another type of protecting group used in peptide synthesis, the structure of which is shown in FIG. 2C. As shown in FIG. 2C, so-called π-stacking (or π-π interactions) occurs between the fluorenyl groups on the Fmoc aromatic groups attached to peptides (amides). It will be appreciated that similar π-stacking will occur between the fluorenyl groups on the Fmoc aromatic groups attached to esters. While the inventors do not wish to be bound by any hypothesis, they believe that such π-stacking between the Fmoc groups enables and encourages hydrogen bonding to occur between the amides or esters in the hydrogel. The inventors believe that such hydrogen bonding between the amides or esters causes the formation of structures, which resemble β-sheets between the plurality of amides or esters in the hydrogel. The inventors believe that these β-sheet-type structures cause the formation of the hydrogel in step (ii) of the method according to the first aspect. Another advantage of Fmoc is that it is thought to have anti-inflammatory properties, which will have significant advantages when the hydrogel is used in medical applications, as will be described hereinafter.

Another preferred aromatic stacking ligand, which may be attached to the amide or ester comprises an aromatic amino acid, i.e. an amino acid residue comprising an aromatic side group (i.e. at least one benzene ring). Examples of suitable aromatic amino acids may include tyrosine, tryptophan, or phenylalanine or less common aromatic amino acids such as di-hydroxy-phenylalanine (DOPA) or other natural or non-natural amino acids with aromatic side chains.

It will be appreciated that preferred hydrogels prepared using the method according to the first aspect of the invention may comprise a plurality of identical peptides, or a plurality of peptides that are different. Nevertheless, in either case, preferably, each peptide in the hydrogel comprises at least two amino acid residues or derivatives or analogues thereof attached to an aromatic stacking ligand, such that interactions therebetween causes the hydrogel to form. The inventors have found that surprisingly, at least two amino acid residues are preferred in each peptide. This is because if a peptide comprises less than two amino acid residues, it results in either no hydrogel forming at all, or an inferior hydrogel being formed, at biologically acceptable conditions.

Peptides of the hydrogel may comprise at least three, four, five, six, or more amino acids or derivatives or analogues thereof, or any combination thereof. However, it is preferred that the peptides may comprise less than 10 amino acids or derivatives or analogues thereof, more preferably less than 8 amino acids or derivatives or analogues thereof, and even more preferably, less than 6 amino acids or derivatives or analogues thereof. Hence, peptides of the hydrogel may comprise at least 2 amino acids and less than 7 amino acids, or derivatives or analogues thereof. For example, the hydrogel may comprise a dipeptide, a tripeptide, a tetrapeptide, a pentapeptide, hexapeptide, and/or a heptapeptide etc., or derivatives or analogues thereof, or any combination thereof.

The hydrogel may comprise a number of identical peptides, a number of peptides that are different from each other, or any combination thereof. Therefore, in one embodiment, the hydrogel may comprise all dipeptides, or all tripeptides, or all tetrapeptides etc. In another embodiment, the hydrogel may comprise a combination of dipeptides and tripeptides, or a combination or tripeptides and tetrapeptides. In yet another embodiment, the hydrogel may comprise a combination of dipeptides, tripeptides, and tetrapeptides, and so on.

Advantageously, smaller peptides such as dipeptides and tripeptides are conveniently small molecules compared to longer peptides (greater than 10 amino acid residues), and are therefore relatively simple and cheap to synthesise. Moreover, due to their small size, dipeptides and tripeptides also exhibit excellent stacking characteristics to thereby form the scaffold under the biologically acceptable conditions.

The inventors have found that the physical properties of the hydrogel formed under biologically acceptable conditions may be altered or ‘tuned’ by choosing different combinations of amino acid residues in the plurality of peptides.

Hence, the amino acids in the plurality of peptides in the hydrogel may be selected from the repertoire of twenty amino acids commonly found in proteins, or any non-naturally occurring amino acids, and the specific amino acids chosen will depend on the final use of the hydrogel, and the condition being treated. For example, the hydrogel may comprise an acidic amino acid, such as aspartic acid, glutamic acid, asparagines, or glutamine; or a basic amino acid, such as histidine, lysine, or arginine. Variation of such amino acids in the peptide will influence the pH of the peptide, and hence, the hydrogel formed. The pH of the hydrogel may therefore be varied depending on the pH of the treatment site.

The hydrogel may comprise a hydrophobic amino acid, such as alanine, cysteine, isoleucine, leucine, methionine, phenylalanine, proline, tryptophan, valine or tyrosine; or a hydrophilic amino acid, such as arginine, asparagine, aspartate, glutamine, glutamate, histidine, lysine, serine, or threonine.

The inventors found that if the peptide comprises two consecutive or adjacent phenylalanine residues, that stable and effective hydrogels are formed. Hence, preferably the peptide comprises at least two consecutive phenylalanine residues.

Therefore, a preferred peptide used in accordance with the invention is Phe-Phe, which is described in the Example. The inventors carried out further investigations as described in the Example, and found that introduction of a further amino acid immediately before the Phe-Phe also formed stable hydrogels. Hence, the inventors produced four tripeptides each of which consisted of Fmoc-X-Phe-Phe, where X=Ala, Val, Leu, Phe. Hence, further preferred peptides include Ala-Val-Phe; Val-Phe-Phe; Leu-Phe-Phe; and Phe-Phe-Phe. In addition, a further preferred peptide, which may be used comprises a mixture of Phe-Phe with Gly-Gly.

In addition, the inventors also made the tripeptide: Fmoc-Leu-Leu-Leu, which also formed stable hydrogels and is also considered a preferred peptide for use in the method according to the invention.

The inventors investigated modifying the peptides in the hydrogel by choosing specific amino acids and combinations thereof. They found that it was possible to tailor the structural and functional characteristics of the resultant hydrogel formed under biologically acceptable conditions. For example, at least one peptide in the hydrogel may comprise at least one amino acid, which is adapted to initiate or promote cell-cell adhesion.

At least one peptide in the hydrogel may comprise an Arginine-Glycine-Aspartate (RGD) peptide motif. The inventors believe that incorporation of the RGD motif (which is a known cell adhesive) will improve the efficacy of the hydrogel to adhere to cells, which would be useful in medical treatments as cell proliferation in the hydrogel will be promoted. Hence, the or each peptide may preferably have the following structure: A.S.L.-AA₁-AA₂-RGD, where A.S.L. denotes the Aromatic Stacking Ligand, where AAₙ denotes amino acid residues in the peptide, and where RGD denotes the RGD motif. It will be appreciated that the above structure is a pentapeptide.

At least one peptide in the hydrogel may comprise an Isoleucine-Lysine-Valine-Alanine-Valine (IKVAV) peptide motif. The inventors believe that incorporation of the IKVAV motif (which is known to directionally guide nerve cells) will improve the efficacy of the hydrogel to guide nerve cells, which would be useful in medical treatments when involving nerve growth, wound repair or nerve tissue regeneration. Hence, the or each peptide may preferably have the following structure: A.S.L.-AA₁-AA₂-IKVAV, where A.S.L. denotes the Aromatic Stacking Ligand, where AAₙ denotes amino acid residues in the peptide, and where IKVAV denotes the IKVAV motif. It will be appreciated that the above structure is a heptapeptide.

At least one peptide in the hydrogel may comprise Lysine-Proline-Valine (KPV) motif. The inventors believe that incorporation of the KPV motif (which has anti-inflammatory properties) will improve the efficacy of the hydrogel as inflammation may occur in the treatment site. Hence, the or each peptide may preferably have the following structure: A.S.L.-AA₁-AA₂-KPV, where A.S.L. denotes the Aromatic Stacking Ligand, where AAₙ denotes amino acid residues in the peptide, and where KPV denotes the KPV motif. It will be appreciated that the above structure is a pentapeptide.

The inventors were surprised to observe that if a peptide in the hydrogel includes an aromatic amino acid, such as phenylalanine, then this resulted in the formation of effective hydrogels under biologically acceptable conditions. This is illustrated by the efficacy of the Phe-Phe tripeptides investigated. Hence, preferably, at least one peptide of the composition used in the method according to the invention comprises at least one aromatic amino acid. By the term “aromatic amino acid”, we mean an amino acid comprising a benzene ring in its side chain.

Preferably, more than one of the peptides of the hydrogel comprises at least one aromatic amino acid. Preferably, the or each peptide comprises a plurality of aromatic amino acids. In preferred embodiments, each amino acid of each peptide in the composition is an aromatic amino acid. Therefore, by way of example, in embodiments where the hydrogel comprises a dipeptide, the dipeptide preferably comprises two aromatic amino acids, and where the hydrogel comprises a tripeptide, the tripeptide preferably comprises three aromatic amino acids.

Examples of suitable aromatic amino acids, which could be included in each peptide in the hydrogel include tyrosine, tryptophan, or phenylalanine. However, it is most preferred that the aromatic amino acid in the peptide comprises phenylalanine. While the inventors do not wish to be bound by any hypothesis, they believe that aromatic amino acids comprising an aromatic side chain contribute to side branching between the peptides in the hydrogel. The inventors believe that such side branching considerably enhances the generation of the hydrogel under biologically acceptable conditions, and this produces an improved scaffold for supporting cell tissues.

Accordingly, it is preferred that the hydrogel comprises a plurality of peptides, or derivatives, or analogues thereof, wherein each peptide comprises at least two amino acid residues, and an aromatic stacking ligand, wherein at least one amino acid comprises an aromatic side chain, and wherein under biologically acceptable conditions, interactions between the stacking ligands cause the hydrogel to form a hydrogel. It is preferred that the amino acid comprising an aromatic side chain is phenylalanine.

Derivatives or analogues of the peptide hydrogel according to the invention may include derivatives or analogues that increase or decrease the peptide's half-life in vivo. Examples of derivatives or analogues capable of increasing the half-life of the peptide according to the invention include peptoid derivatives, D-amino acid derivatives of the peptides, and peptide-peptoid hybrids.

The peptide used in the invention may be subject to degradation by a number of means (such as protease activity in biological systems). Such degradation may limit the bioavailability of the peptide, and hence the ability of the peptide to achieve its biological function. There are wide ranges of well-established techniques by which peptide derivatives or analogues that have enhanced stability in biological contexts can be designed and produced. Such peptide derivatives may have improved bioavailability as a result of increased resistance to protease-mediated degradation.

Preferably, a peptide derivative or analogue suitable for use according to the invention is more protease-resistant than the peptide from which it is derived. Protease-resistance of a peptide derivative and the peptide from which it is derived may be evaluated by means of well-known protein degradation assays. The relative values of protease resistance for the peptide and the peptide derivative or analogue may then be compared.

Peptoid derivatives of the peptide hydrogel used in the invention may be readily designed from knowledge of the structure of the peptide. Peptoid compounds have two properties that make them suitable for use as peptide derivatives/analogues according to the invention:—
(i) In peptoid residues, no hydrogen bond involving the NH would be possible.
(ii) The peptoids are resistant to enzymatic degradation.

Commercially available software may be used to develop peptoid derivatives according to well-established protocols.

Retropeptoids, (in which all amino acids are replaced by peptoid residues in reversed order) are also able to mimic peptides. A retropeptoid is expected to bind in the opposite direction in the ligand-binding groove, as compared to a peptide or peptoid-peptide hybrid containing one-peptoid residue. As a result, the side chains of the peptoid residues are able to point in the same direction as the side chains in the original peptide.

Hence, it is preferred that the hydrogel comprises a plurality of peptides, or derivatives, or analogues thereof, wherein each peptide comprises at least two amino acid residues attached to Fmoc.

Preferably, under biologically acceptable conditions, interactions between the Fmoc structures cause the formation of the hydrogel. A preferred peptide is Fmoc-Phe-Phe. Another preferred peptide comprises a mixture of Fmoc-Phe-Phe and Fmoc-Gly-Gly.

Another preferred aromatic stacking ligand, which may be attached to the peptide in the hydrogel used, comprises an aromatic amino acid, i.e. an amino acid residue comprising an aromatic side group (i.e. at least one benzene ring). Accordingly, in this embodiment, because the aromatic stacking ligand is itself an aromatic amino acid, and because it is attached to at least two other amino acid residues, the hydrogel comprises at least three amino acid residues. Where the ligand is an aromatic amino acid attached to a tripeptide, the hydrogel comprises a tetrapeptide, and so on.

Examples of suitable aromatic amino acids may include tyrosine, tryptophan, or phenylalanine, or less common aromatic amino acids such as di-hydroxy-phenylalanine (DOPA), or other natural or non-natural amino acids with aromatic side chains. Hence, the hydrogel used in the method according to the invention may comprise a plurality of peptides, or derivatives, or analogues thereof, wherein each peptide comprises at least two amino acid residues attached to an aromatic amino acid residue.

In addition to the peptides, which comprise at least two amino acid residues, in the hydrogel, the inventors also investigated modifying the hydrogel by adding further components thereto. They added various additives to the hydrogel components, and found that it was possible to further tailor the structural and functional characteristics of the resultant hydrogel formed under biologically acceptable conditions, such characteristics depending on the intended use of the hydrogel. Therefore, the hydrogel may further comprise a bioadditive.

By the term “bioadditive”, we mean a compound exhibiting biologically active functionality.

By way of example, the bioadditive may be adapted to promote or improve cell adhesion. It is known that cells respond favourably to positive charges. Hence, it is preferred that the bioadditive is positively charged. The bioadditive may comprise at least one further amino acid, or a peptide. Therefore, the bioadditive may comprise a positively charge amino acid residue, for example, arginine, histidine, or lysine. The inventors have demonstrated in the Examples that the addition of lysine (K) significantly improves cell adhesion.

It is preferred that the bioadditive itself comprises an aromatic stacking ligand, which may be provided so that the bioadditive is able to form hydrogen bonds with the peptides of the hydrogel used according to the invention. Suitable aromatic stacking ligands, are as described hereinbefore. Hence, a preferred aromatic stacking ligand comprises Fmoc. As mentioned herein, Fmoc is thought to have anti-inflammatory properties. In another embodiment, the bioadditive may be Fmoc.

Hence, the bioadditive may preferably have the following structure: A.S.L.-K, where A.S.L. denotes the Aromatic Stacking Ligand, and where K denotes the Lysine residue. It will be appreciated that the above structure is a single amino acid attached to the aromatic stacking ligand. Preferably, the aromatic stacking ligands comprise Fmoc.

Hence, a preferred peptide used in the method in accordance with the invention comprises a mixture of Fmoc-Phe-Phe with Fmoc-Lys.

The inventors of the present invention focussed their research on using the hydrolase catalysed method according to the first aspect to prepare hydrogels according to the second aspect, which hydrogels comprised self-assembling peptides, as they believed that these could be used to form a scaffold that mimics the extracellular matrix (ECM) of certain tissues. The inventors wanted to investigate if these enzymatically produced hydrogels would be capable of supporting individual cells and cell cultures under biologically acceptable conditions, i.e. stable under in vivo tissue culture conditions of high ionic strength, and a neutral pH. As a model cell culture, the inventors focussed their research on supporting cultures of chondrocytes (cartilage cells) on the hydrogel scaffold.

The inventors therefore produced a series of tripeptides, each of which consisted of Fmoc-X-Phe-Phe, where X=Alanine, Valine, Leucine, Phenylalanine. In addition, the inventors also made the tripeptide: Fmoc-Leu-Leu-Leu. Each of these tripeptides were produced by the method according to the first aspect, i.e. reacting an Fmoc amino acid with a dipeptide and then contacting the mixture with a hydrolase enzyme. The inventors were surprised to see that each of these five tripeptides were able to self-assemble into a hydrogel in a physiological buffer under biologically acceptable conditions (pH=7.0). To date, this had not been possible.

Hence, preferred peptides used in hydrogel may comprises Fmoc-X-Phe-Phe, where X=Alanine, Valine, Leucine, or Phenylalanine. A further preferred peptide comprises Fmoc-Leu-Leu-Leu.

Surprised to find that these tripeptides were able to produce stable hydrogels under physiological conditions, the inventors decided to carry out further experiments. They also found that the hydrogels formed by the self-assembled peptides as described herein are surprisingly adapted to support cell cultures therein as described in the examples.

Hence, in summary, the inventors have surprisingly demonstrated that hydrolase enzymes may be used to prepare hydrogels, formed from self-assembling peptides that are:—(i) stable under biologically acceptable, tissue culture conditions; (ii) are of similar dimensions to fibrous components of the extracellular matrix (i.e. nano-sized fibres); and (iii) are capable of supporting cell culture in both 2D and in 3D. Hence, advantageously, the inventors believe that the hydrogels formed by such self-assembling Fmoc-dipeptides may be used in a wide range of medical applications, for example, in tissue engineering and regeneration scenarios, and in methods of treatment.

Therefore, according to a third aspect of the present invention, there is provided a method of treating an individual suffering from a medical condition characterised by tissue loss/damage, the method comprising providing at a treatment site of an individual in need of such treatment, a hydrogel according to the second aspect.

It will be appreciated that the hydrogel used in the method according to the third aspect may be comprised of a plurality of esters, each ester comprising an aromatic stacking ligand. However, it is preferred that the hydrogel used in the method according to the third aspect may be comprised of gel-forming peptides, or derivatives, or analogues thereof, wherein each peptide comprises at least two amino acid residues and an aromatic stacking ligand.

The inventors have surprisingly found that the use of self-assembling peptides to form a hydrogel at the treatment site enables the formation of a hydrogel scaffold structure, which is adapted to support cell growth. The inventors observed that the cells are able to infiltrate the hydrogel at the treatment site, thereby forming a 3D cell culture. This cell culture therefore can replace and/or repair the tissue lost or damaged at the treatment site.

The self-assembling subunits of the peptide hydrogel (ie. the gel-forming peptides, derivatives or analogues thereof) may have a molecular weight of between 100 and 20,000 Da, more preferably, between 200 and 15,000 Da, and even preferably, between 300 and 12,000 Da.

In one embodiment, the hydrogel may be formed remote from the treatment site, for example, in a mould; which may then be administered to the treatment site. The choice of how to administer the hydrogel to the treatment site will depend on the medical condition being treated. In either case, the hydrogel may be used as a scaffold structure to support cells therein, to thereby repair the site of tissue loss or damage.

In one embodiment, the hydrogel may be provided as a liquid precursor composition, which may then be induced in situ to form the hydrogel. Hence, the hydrogel may be prepared in situ in the treatment site. 2-3% of expressed proteins in mammals are thought to be proteases and so the inventors believe that these may be harnessed by forming the hydrogel in situ. It will be appreciated that different hydrolases in mammals will recognise different amino acid sequences. Hence, the first and second molecules (precursors) used may be chosen such that they are reacted together by a specific enzyme in the individual being treated. The inventors believe that the first and second molecules used in step (i) of the method of the first aspect may be introduced into the treatment site, exposed to a hydrolase (preferably a protease) in the treatment site, which induces hydrogel formation. Additional hydrolase may be added to the precursor molecules if required. A suitable hydrolase may be thermolysin or chymotrypsin.

The liquid precursor composition preferably comprises the first molecule comprising the carboxylic acid and the second molecule comprising the amine used in step (i) of the method according to the first aspect.

Hence, the inventors believe that the method according to the third aspect, may be used in wide variety of different medical treatments for treating a medical condition characterised by tissue loss/damage. Examples of conditions that may be treated include the treatment of wounds, and related injuries, and tissue degenerative disorders. For example, the wound may be chronic, and may be abrasive, for example, burns. The wound may be formed by pressure, such as decubitus ulcers, and bed-sores. The wound may be acute, and may be penetrative such as a cut, or a stab wound, or the result of a crush to the body of the individual requiring treatment.

Tissue degenerative disorders that may be treated using the method include neurodegenerative, intervertebral disc disorders, cartilage or bone degeneration such as osteoarthritis, osteoporosis, liver degenerative disorders, kidney degenerative disorders, muscle atrophy.

Preferably, and advantageously, the peptides, or derivatives, or analogues thereof used in the method according to the third aspect of the invention may be induced to form a hydrogel. The hydrogel is preferably optically transparent, which is an advantage for medical practitioners to clearly see the treatment site when using the hydrogel in the method. It is preferred that the hydrogel is provided in a physiologically acceptable excipient. By the term “physiologically acceptable excipient”, we mean any suitable solution, which is capable of conferring biologically acceptable conditions on the peptides such that they self-assemble (i.e. with each other) resulting in gelation to form the hydrogel. Examples of suitable excipients will be known to the skilled technician, and may comprise a physiological buffer, such as saline. Preferably, the excipient is provided at a biologically acceptable pH.

Hence, the inventors have demonstrated for the first time that peptides, derivatives or analogues thereof may be contained within a physiologically acceptable excipient, such that the peptides which are attached to an aromatic stacking ligand, self-assemble to form the hydrogel. Hence, preferably, the excipient confers biologically acceptable conditions on the peptides, derivatives or analogues thereof, such that interactions between the stacking ligands cause the peptides, derivatives or analogues thereof to form a hydrogel either in the treatment site, or prior to administration thereto.

Previous researchers have only demonstrated preparation of hydrogels under non-physiological (i.e. biologically unacceptable) conditions, for example, where the pH is substantially low and therefore acidic. Hence, to date, it has not been possible to form hydrogels at biologically acceptable pH's. Hence, the prior art does not contemplate the use of such hydrogels in medical contexts, as it will be appreciated that acidic conditions will be wholly unsuitable for biological applications of the hydrogel used in the method according to the invention. Therefore, the inventors believe that use of the hydrogel in the method of the invention is a significant advance over current technology.

It is preferred that the biologically acceptable excipient is at a pH of between 5 and 9, more preferably between 6 and 8, even more preferably, between about 6.5 and about 7.5. It will be appreciated that the pH of most cells is about 7.4. Hence, a most preferred excipient has a pH of between about 7 and about 7.5. It will be appreciated that such pHs are referred to as being biologically acceptable conditions.

By the term “biologically acceptable conditions”, we mean the hydrogel used in the method of the invention is substantially stable under in vivo conditions, i.e. conditions of pH, ionic strength and temperature, which would be found in vivo. The inventors envisage primarily using the method according to the third aspect of the invention, and hence, the hydrogel to treat disorders characterised by tissue damage/loss in mammals and, in particular, man. Therefore, it is preferred that the hydrogel is formed and is stable under biologically acceptable conditions in mammals, and preferably, in man.

Hence, the inventors investigated the stability of the hydrogel at a biologically acceptable pH. Since the inventors envisage primarily using the hydrogel in mammals, they considered a biologically acceptable pH at which the hydrogel should be stable to be between about 5.0 to about 9.0. The inventors believe that the treatment site in the disorders being treated would be within this pH range. However, it is preferred that the hydrogel is formed at a pH of between about 6.0 to about 8.0. As described herein, the method may be used to treat wounds. In chronic wounds, the pH may be between a 6.0 and 8.0. Hence, when treating chronic wounds, it is preferred that the hydrogel is stable between a pH of about 6.0 and 8.0.

However, when treating other disorders, the hydrogel may be formed at a pH of between about 6.5 to about 7.5. It is more preferred that the hydrogel is formed at a pH of between about 6.7 to about 7.3, and still more preferably, between about 6.9 to about 7.1. It will be appreciated that it is most preferred that the hydrogel is formed at about pH 7.0. It is preferred that the hydrogel is substantially stable at these biologically acceptable pH's.

The inventors also investigated the stability of the hydrogel under biologically acceptable ionic conditions. The inventors believe that the treatment site of the individual being treated would be at a high ionic strength. Hence, it is preferred that the hydrogel is formed in conditions of substantially high ionic strength. Hence, the ionic strength may be between about 0.01M to about 1M, preferably, between about 0.05M to about 0.5M, more preferably, between about 0.1 to about 0.2, and even more preferably, between about 0.12M and about 0.17M.

Furthermore, the inventors investigated the stability of the hydrogel at biologically acceptable temperatures. Since the inventors envisage primarily using the hydrogel in the method of the third aspect to treat mammals and in particular man, they considered biologically acceptable temperatures to be between about 32° C. to about 40° C. Hence, it is preferred that the hydrogel used in the method is substantially liquid at temperatures above about 40° C.

The inventors were surprised to find that it was possible to tightly control the gelation of the hydrogel at temperatures below 40° C. In fact, they found that the critical gelation temperature for the hydrogel was at about body temperature (i.e. 37° C. and below), and that the gel liquefies at temperatures greater than body temperature. This is a major advantage for use of the hydrogel in medicine, as it is therefore possible to induce transition of the peptides from liquid form (sol) to hydrogel (gel) on demand when in situ in the treatment site. Hence, preferably, the hydrogel used in the method is formed below about 40° C., more preferably below about 39° C., and even more preferably, below about 38° C. Therefore, preferably, the hydrogel is formed at a temperature of between about 36° C. to about 38° C., and most preferably, at about 37° C.

However, it should be appreciated that in chronic wounds, and also in surface organs (such as the skin, the eye etc.) the temperature may be a few degrees lower, for example, about 32° C. to 34° C. Hence, in embodiments of the method where the composition is used to treat chronic wounds or surface organs, it is preferred that the hydrogel forms at a temperature of between about 32° C. to 34° C.

Therefore, in preferred embodiments of the invention, it is preferred that the hydrogel forms at a pH of between about 6.8 to about 7.5, a high ionic strength, and at a temperature of between about 32° C. to about 38° C.

It will be appreciated that the hydrogel may be either used effectively in a number of different physical forms. For example, in one embodiment, the method may comprise administering to the treatment site a liquid hydrogel precursor composition in the form of a solution, which may then be induced to form the hydrogel, by the action of the hydrolase. Alternatively, in another embodiment, the method may comprise administering to the treatment site the already formed hydrogel composition. The inventors believe that each of these embodiments is an important aspect of the invention, which may be used with the method of the first aspect.

Hence, in a fourth aspect, there is provided a liquid hydrogel precursor composition comprising a first molecule comprising a carboxylic acid group; a second molecule comprising an amine group or an alcohol group; and a hydrolase enzyme, wherein at least one of the first or second molecules comprises an aromatic stacking group

Preferably, the hydrogel precursor composition may be induced to form a hydrogel, upon action of the hydrolase.

In a fifth aspect, there is provided a hydrogel according to the second aspect and a physiologically acceptable excipient for use as a medicament.

The hydrogel is preferably fibrous, and the self-assembly of the hydrogel is preferably caused by interactions between the stacking ligands attached to each peptide. Furthermore, advantageously, by choosing specific amino acid residues, which make up the plurality of peptides, it is possible to vary the structural and functional properties of the hydrogel formed. Therefore, the peptides and hence the hydrogel may be specifically ‘tailored’, depending on the final use of the hydrogel. In addition, such tailoring will involve choosing which hydrolase may cause the hydrogels precursor molecules to react.

As mentioned herein, prior art hydrogels have only been made at acidic pH, and it will be appreciated that low pHs are unsuitable for medical applications. Therefore, because the hydrogel according to the invention forms in a physiological excipient under biologically acceptable conditions, the inventors wanted to assess whether functional cues or moieties could be incorporated into the hydrogel's structure so that they could be adapted for medical uses. The inventors therefore tested the hydrogel formed from Fmoc-Phe-Phe (and mixtures therewith) for its stability in cell culture conditions, and its ability to support cell cultures or tissues. As discussed in the Examples, the hydrogels tested had the surprising ability to organise cells into a three-dimensional architecture. The inventors have therefore observed that the hydrogels according to the invention are surprisingly suitable for culturing and supporting cells therein. The inventors then conducted statistical analysis of data used in an MTT Assay, which further confirmed the surprising finding the cell growth actually continued for the entire time measured, i.e. up to 7 days.

Therefore, it is preferred that the hydrogel according to the second aspect or the hydrogel used in the method according to the third aspect, or the precursor composition is adapted to support at least one cell, to thereby form a physiologically stable cell-supporting medium or cell scaffold. Hence, the hydrogel or the composition may be seeded with at least one cell.

Hence, according to a sixth aspect of the present invention, there is provided a cell-supporting medium comprising the hydrogel according to the second aspect, or the composition of the fourth aspect, and at least one cell.

The cell-supporting medium of the sixth aspect may be referred to as a cell-hydrogel scaffold. Preferably, the cell-supporting medium is adapted to support a plurality of cells. Preferably, the or each cell is biochemically functional in vivo. Accordingly, the plurality of cells may form a cell culture or a tissue.

As the hydrogel precursor composition in the fourth aspect is a liquid, at least one cell may be suspended therein.

The inventors investigated various methods for preparing the cell-supporting medium according to the sixth aspect.

Hence, in a seventh aspect, there is provided a method of preparing a cell supporting medium according to the sixth aspect, the method comprising the steps of:—
(i) contacting either a hydrogel of the second aspect, or a liquid hydrogel precursor composition according to the fourth aspect, with at least one cell; and
(ii) exposing the hydrogel or composition to conditions such that the at least one cell is supported on and/or in a hydrogel, thereby forming a cell-supporting medium.

It will be appreciated that the method according to the seventh aspect may be carried out in situ in the treatment site, or remote from the treatment site, and then transferred thereto.

The skilled technician will appreciate how to culture various cell types with the hydrogel or compositions according to the invention. Hence, it will be appreciated that the specific details of the methodologies (culture time, temperatures, growth media etc) used will depend on the type of cell involved, and the final use of the cell-supporting medium (ie. the scaffold). By way of the example, the Example provides details of how to culture chondrocytes and to produce a chondrocyte cell scaffold.

In one embodiment, step (i) of the method according to the invention may comprise contacting the liquid hydrogel precursor composition according to the invention with the at least one cell. In another embodiment, step (i) of the method according to the seventh aspect may comprise contacting the hydrogel composition according to the invention with the at least one cell. The nature of step (ii) of the method will be determined by whether the composition in step (i) is in liquid form or a hydrogel.

Hence, in one embodiment, the method may comprise exposing the precursor composition of the invention to conditions such that a hydrogel is formed in step (i) prior to contacting the at least one cell therewith. Such conditions may comprise lowering the temperature of the composition to below the critical gelation temperature, e.g. less than 40° C. The inventors investigated this embodiment of the method, and surprisingly found that cells in a culture media were rapidly taken up by the hydrogel in step (ii) of the method to form the cell-supporting medium. They found that the cell culture distributed itself on and throughout the hydrogel in step (ii). The inventors envisage that this embodiment will have great utility in the method of the third aspect.

In an alternative embodiment, the composition may be initially maintained under conditions in which it is in the form of the liquid precursor in step (i) of the method, to which the at least one cell is added in step (ii). Hence, the method may comprise initially exposing the composition in step (i) to conditions in which it is substantially liquid (i.e. not a hydrogel). For example, the composition may be exposed to a pH or temperature or ionic strength at which the compound is liquid. For example, the composition may be exposed to a temperature above the critical gelation temperature of about 40° C. or more, such that it liquifies. The method may then comprise the step of contacting the at least one cell with the liquid precursor in step (i). After step (i), step (ii) preferably comprises exposing the liquid precursor composition to conditions in which it forms a hydrogel, preferably the action of the hydrolase enzyme. The temperature may be cooled to about 37° C., or the pH may be adjusted such that the hydrogel is preferably formed with cells distributed throughout. The hydrogel which forms, in which the at least one cell is supported is referred to as the cell-supporting medium or cell scaffold. Again, the inventors believe that this embodiment will have great utility in the method of the third aspect.

The hydrogel or composition according to the invention, or the medium according to the sixth aspect may be used in a number of ways. A common problem with many wounds or tissue degenerative disorders is that a cavity or space may be formed in the body of the individual being treated, and this cavity or space will need to be repaired using the composition of cell support medium. Hence, the composition or medium may be prepared either in vitro or in vivo. Furthermore, the composition or medium may be prepared either: (i) in situ (in the wound itself); or (ii) remote from the wound, and then transferred to the area to be treated after it has been prepared.

Preferably, the method according to the seventh aspect is used to prepare the cell-supporting medium. Therefore, in one embodiment, the hydrogel or composition according to the invention is preferably administered to the area to be treated (wound, cavity, or degenerated area). It will be appreciated that the composition according to the fourth aspect is in liquid form and the hydrogel according to the second aspect is in the form of a hydrogel. Once the composition is in position in the area to be treated, at least one cell is then contacted therewith as in step (i) of the method according to the fifth aspect. If the composition is a hydrogel already, then at least one cell can be contacted therewith to allow the cell scaffold to form. If the compound is in the form of the liquid hydrogel precursor, then the hydrolase enzyme induces hydrogel to form.

In another embodiment, the cell-supporting medium may be prepared remote from the wound (eg. in the lab), and is then preferably administered to the area to be treated. In this approach, the gel would be formed in a pre-determined three-dimensional shape for example, by using a mould, and cells may either be added prior to the gelation process or after the gel has formed, again by the hydrolase enzyme. The pre-formed gel may then be implanted in the body where the patient's cells migrate into the gel scaffold. Examples of this use would be in tissues, which have a migratory capacity and/or those, which are responsible for tissue remodelling. Examples are skin, bone, and peripheral nerves. The implant may also be supplemented with further cells externally by the medical practitioner. In addition, other factors, which may simulate cell and preferably tissue growth, may be added to the implant, for example, growth factors.

Preferably, the cell supporting medium or hydrogel, whether prepared in situ in the area to be treated, or remote from it, is suitably maintained to allow the at least one cell to divide to form a culture or tissue therein. Accordingly, it will be appreciated that the hydrogel acts as a supporting scaffold for the tissue and thereby allows repair of the wound, or regeneration of the damaged tissue.

The inventors believe that the method according to the third aspect, may be used in wide variety of different, medical treatment methods, such as tissue regeneration/engineering applications, controlled stem cell differentiation, and in wound healing. The types of tissues and wound which could be treated are varied, and hence, it will be appreciated that the invention is not limited to any specific type of cell, which could be supported and cultured on the hydrogel administered to the treatment site. However, by way of example, suitable cells, which may be supported in the hydrogel include epithelial cells (e.g., hepatocytes), neurons, endothelial cells, osteoblasts (bone cells), chondrocytes (cartilage cells), fibroblasts, smooth muscle cells, osteoclasts, keratinocytes, nerve progenitor cells, Schwann cells, stem cells, macrophages, islet cells, and tumour cells, etc.

The cell type contacted with the composition or cell-supporting medium will depend on the type of wound being repaired, or the type of tissue being regenerated. Therefore, by way of example, if the wound is in skin, then at least one skin cell may be contacted with the hydrogel, composition or cell-supporting medium. If the wound is in bone, then at least one bone cell or osteoblast is preferably contacted with the hydrogel, composition or cell-supporting medium. If the wound is in cartilage, then at least one chondrocyte is preferably contacted with the hydrogel, composition or cell-supporting medium. If the eye tissue has been damaged, it may be required to contact the hydrogel, composition or cell-supporting medium with eye stem cells. It will be appreciated that different types of cell type may be contacted with the hydrogel, composition, or cell supporting medium, if necessary.

As discussed in the Examples, the inventors focussed their research on investigating the efficacy of the hydrogel to support cartilage cells. Hence, it is preferred that the at least one cell is a chondrocyte. This would be advantageous, if the treatment site is a site in which cartilage has been damaged or lost. However, the at least one cell may be an osteoblast or bone cell. This would be useful if the site being treated is bone. The osteoblast may be autologous or autogenous.

Alternatively, the at least one cell may be a stem cell, which may be either mesenchymal, or haematopoeic, or embryonic, or cloned. The inventors believe that the ability to culture and support a wide variety of cells such as chondrocytes, osteoblasts and stem cells, will be of significant importance in many aspects of medicine.

The method according to the third aspect may comprise use of the hydrogel or composition or cell-supporting medium according to the fourth aspect. The hydrogel, composition or cell supporting medium may be combined in formulations having a number of different forms depending, in particular on the manner in which the formulation is to be used. It will be appreciated that the vehicle of the composition of the invention should be one which is well-tolerated by the subject to whom it is given, and preferably enables efficient delivery of the composition to a target site. Thus, for example, the composition may be in the form of a liquid (composition according to the second aspect), or gel or hydrogel (composition according to the third aspect), or any other suitable form that may be administered to a person or animal.

The inventors believe that the Fmoc peptides described herein may be formulated with a physiologically acceptable excipient to form a medicament. The inventors believe that the prior art does not hint at or even suggest that hydrogels according to the invention may be used as a medicament.

Therefore, according to a further aspect of the invention, there is provided a composition comprising a first molecule comprising a carboxylic acid group; a second molecule comprising an amine group or an alcohol group; a hydrolase enzyme; and at least one cell, wherein at least one of the first or second molecules comprises an aromatic stacking group, for use as a medicament.

In particular, the inventors envisage the medicament having major uses in a wide variety of tissue engineering and regeneration applications, and also in wound healing. Such disorders are commonly linker in that they a characterised by tissue damage or loss.

Therefore, according to a further aspect, there is provided use of a composition comprising a first molecule comprising a carboxylic acid group; a second molecule comprising an amine group or an alcohol group; a hydrolase enzyme; wherein at least one of the first or second molecules comprises an aromatic stacking group, for the preparation of a medicament for the treatment of a medical condition characterised by tissue loss/damage.

It will be appreciated that the medicament may be used to treat individuals suffering from a wide variety of disease conditions characterised by tissue loss or damage. Examples include wounds and/or tissue degenerative disorders.

The wound may be chronic or acute. Tissue degenerative disorders that may be treated include neurodegenerative, intervertebral disc disorders, cartilage or bone degeneration such as osteoarthritis, osteoporosis, liver degenerative disorders, kidney degenerative disorders, muscle atrophy.

It will be appreciated that in chronic wounds, it has been described that modulating the pH of the wound may help improve wound healing. The pH in chronic wounds varies between 6 and 8, and the inventors believe that wound healing appears to work best at reduced pH values. Hence, the composition may comprise acidic or basic amino acids (His, Arg, Lys, Glu, Asp), which may help maintain the pH of the hydrogel in the treatment site.

Furthermore, in chronic wounds, the temperature may be a few degrees lower than normal body temperature, ie. about 32° C. to 34° C. Furthermore, for treating surface organs such as the eye, skin, and so on, etc the preferred temperature will be lower than normal body temperature. However, the composition will need to gel at this temperature range to form the scaffold.

It will be appreciated that the hydrogel, compositions, or the cell-supporting medium according to the invention may be used to formulate the medicaments of the invention. Furthermore, the medicament may be used in the method of treatment according to the third aspect.

The hydrogel, compositions, cell-supporting medium, or medicament according to the invention may be used in a monotherapy (i.e. use of the hydrogel, composition, cell supporting medium, or medicament, alone). Alternatively, the hydrogel, compositions, cell-supporting medium, or medicament according to the invention may be used as an adjunct, or in combination with other known therapies.

In some circumstances, the composition, compound or scaffold according to the invention may be administered by injection into the wound areas. Injections may be intravenous (bolus or infusion) or subcutaneous (bolus or infusion).

The hydrogel, compositions, cell-supporting medium, or medicament may also be incorporated within a slow or delayed release device. Such devices may, for example, be positioned on or adjacent the area to be treated, for example by implantation, and the hydrogel, compositions, cell-supporting medium, or medicament may be released over weeks or even months. Such devices may be particularly advantageous when long-term treatment with the medicament is required and which would normally require frequent administration (e.g. at least daily injection or implant).

It will be appreciated that the amount of hydrogel, compositions, cell-supporting medium, or medicament according to the invention required will be determined by its biological activity and bioavailability, which in turn depends on the mode of administration, the physicochemical properties of the medicament employed, and whether the hydrogel, compositions, cell-supporting medium, or medicament is being used as a monotherapy or in a combined therapy. The frequency of administration will also be influenced by the above-mentioned factors and particularly the half-life of the medicament within the subject being treated.

Optimal dosages to be administered may be determined by those skilled in the art, and will vary with the particular medicament in use, the strength of the preparation, the mode of administration, and the advancement of the disease condition. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials, etc.), may be used to establish specific formulations of the medicament according to the invention, and precise therapeutic regimes (such as daily doses and the frequency of administration).

Generally, a daily dose of between 0.01 μg/kg of body weight and 1.0 g/kg of body weight of the hydrogel according to the invention may be used for the prevention and/or treatment of the specific medical condition. More preferably, the daily dose is between 0.01 mg/kg of body weight and 100 mg/kg of body weight. Daily doses may be given as a single administration (e.g. a single daily tablet). Alternatively, the medicament may require administration twice or more times during a day. As an example, the medicament according to the invention may be administered as two (or more depending upon the severity of the condition) daily doses of between 25 mg and 5000 mg. A patient receiving treatment may take a first dose upon waking and then a second dose in the evening (if on a two dose regime) or at 3 or 4 hourly intervals thereafter. Alternatively, a slow release device may be used to provide optimal doses to a patient without the need to administer repeated doses.

The invention further provides a pharmaceutical composition comprising a therapeutically effective amount of a hydrogel, compositions, cell-supporting medium, or medicament according to the invention. In one embodiment, the amount of the hydrogel is an amount from about 0.01 mg to about 800 mg. In another embodiment, the amount of the hydrogel is an amount from about 0.01 mg to about 500 mg. In another embodiment, the amount of the hydrogel is an amount from about 0.01 mg to about 250 mg. In another embodiment, the amount of the hydrogel is an amount from about 0.1 mg to about 60 mg. In another embodiment, the amount of the hydrogel is an amount from about 0.1 mg to about 20 mg.

The invention also provides a process for making a pharmaceutical composition, the process comprising combining a therapeutically effective amount of a hydrogel, compositions, or cell-supporting medium according to the present invention, and a pharmaceutically acceptable vehicle. A “therapeutically effective amount” is any amount which, when administered to a subject provides prevention and/or treatment of a specific medical condition. A “subject” may be a vertebrate, mammal, domestic animal or human being.

A “pharmaceutically acceptable vehicle” as referred to herein is any physiological vehicle known to those of ordinary skill in the art useful in formulating pharmaceutical compositions. The pharmaceutically acceptable vehicle may be a liquid, and the pharmaceutical composition is in the form of a solution. In a further preferred embodiment, the pharmaceutical vehicle is a gel or hydrogel, and the composition is in the form of a cream or the like. In both cases, the composition may be applied to the treatment site.

The composition may comprise one or more substances, which may also act as lubricants, solubilisers, suspending agents, fillers, glidants, compression aids, or binders. It can also be an encapsulating material. Liquid vehicles are used in preparing solutions, suspensions, emulsions, syrups, elixirs and pressurized compositions. The hydrogel, compositions, cell-supporting medium, or medicament may be dissolved or suspended in a pharmaceutically acceptable liquid vehicle such as water, an organic solvent, a mixture of both or pharmaceutically acceptable oils or fats. The liquid vehicle may contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration and implants include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellent.

In cases where it is desired to inject or implant the hydrogel, compositions, cell-supporting medium, or medicament directly to the treatment site, liquid pharmaceutical compositions which are sterile solutions or suspensions can be utilized by for example, intramuscular, intrathecal, epidural, intraperitoneal, intravenous and particularly subcutaneous, intracerebral or intracerebroventricular injection. The hydrogel may be prepared as a sterile hydrogel composition that may be dissolved or suspended at the time of administration using sterile water, saline, or other appropriate sterile injectable medium. Vehicles are intended to include necessary and inert binders, suspending agents, lubricants, sweeteners, preservatives, dyes, and coatings.

It is preferred that the hydrogel, compositions, cell-supporting medium, or medicament according to the invention may be implanted in the form of a sterile solution or suspension or gel or hydrogel containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. Preferably, the hydrogel is implanted either in liquid or solid (hydrogel) composition form. Compositions suitable for implants include liquid forms, such as solutions, syrups, elixirs, and suspensions.

It will be appreciated that the self-assembling hydrogels according to the invention have a wide range of medical applications, for use in the method of the first aspect. In addition, the inventors also explored the use of the self-assembling hydrogel in a range of non-medical applications, for example, in 3D cell culturing, in vitro toxicity testing, understanding cell/extracellular matrix interactions, studies of mechanical loading effects on cells, and cell study or metastasis models.

Therefore, the inventors made a comparison of current materials, which are available for in vitro 3D cell studies, with the hydrogel used in the method according to the first aspect of the invention. The currently available materials that the inventors tested included: Puramatrix; Bovine Collagen; Agarose; and chitosan.

Hence, according to a further aspect, there is provided use of a composition according to the cell-supporting medium according to the fourth aspect for studying a cell culture in vitro.

The comparison showed that Puramatrix can be somewhat difficult to handle, and initially somewhat toxic to cells (pH 3-4). Furthermore, bovine collagen, agarose and chitosan are unsatisfactory model systems due to batch to batch variations of the material, difficulty in handling and/or significantly different properties to the in vivo extracellular matrix. It is preferred that the use comprises initially preparing a hydrogel from the self-assembling peptides, and then adding a cell culture thereto, so that the cell behaviour under conditions that mimic in vivo growth environment can be studied. Hence, preferably the cell culture grown on the cell-supporting medium is substantially 3D. The growth experiments may be carried out in 20 or 96 well plate format and may have applications in 3D cell culture, in vitro toxicity testing, understanding cell/extracellular matrix interactions, controlled stem cell differentiation, studies of mechanical loading effects on cells, and the study of metastasis models. Currently, Puramatrix, Bovine Collagen, Agarose or chitosan are used, which the inventors have found to be significantly inferior cell supporting medium according to the fourth aspect.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

FIG. 1 shows an overall equilibrium of enzyme triggered self assembly of amides or esters in accordance with the method of the invention. Fmoc-AA₁-COO⁻ is a fluorenylmethoxycarbonyl (Fmoc) N-protected amino acid that acts as an acyl donor, and H₃N⁺-AA₂-AA₃-COO⁻ is a dipeptide, acting as a nucleophile. Whilst equilibrium K_{eq}1 lies toward hydrolysis of the tri-peptide, K_{eq}2 is expected to lie towards gelation for a number of peptides, driven by the π-stacking of Fmoc groups. In cases where K_{eq}2 is sufficiently larger than K_{eq}1 the overall reaction is expected to proceed from the non-gelling precursors to the self assembled hydrogel;

FIG. 2 shows a proposed self assembly mechanism: (A): (1) Fmoc amino acids (grey) are enzymatically coupled to di-peptides (black) by a protease (i) to form Fmoc-tripeptides (2). If the thermodynamics are sufficiently favourable, these Fmoc-tripeptides self-assemble (ii) through π-π interactions between highly conjugated Fmoc groups. These stacks in turn associate to form nanoscopic fibres (4). (B): Chemical structures of Fmoc amino acids, di-peptides and the amino acid side chains R. (C). Proposed self assembly mechanism: the Fmoc-tri-peptides stack through π-π interactions, peptide side chains are stabilized through hydrogen bonding.

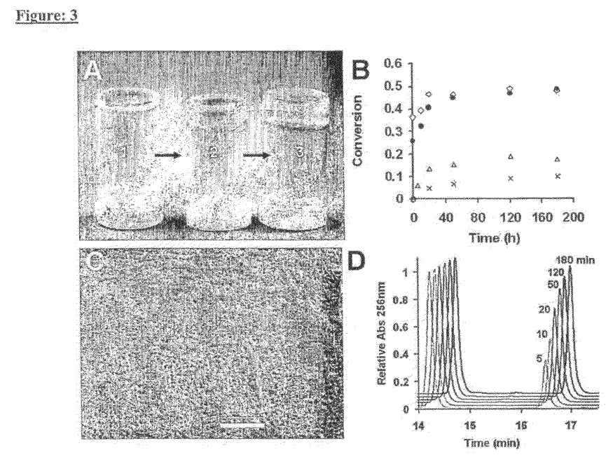

FIG. 3 shows A: 1 mixture of Fmoc-Phe and Phe-Phe, 2, addition of enzyme, 3, formation of transparent self assembled hydrogel within 5 minutes. B: Effect of enzyme amount on the rate of gel formation. From top to bottom they represent 2, 0.5, 0.1, 0.05 mg thermolysin, respectively. Conversions are based on the HPLC peak areas at 256 nm. C: Cryo-SEM micrograph of enzymatically prepared Fmoc-(Phe)₃ self assembled peptide hydrogel. This gel was obtained from 40 μmol of Fmoc-Phe-OH, 40 μmol of H-Phe-Phe-OH and 0.5 mg of thermolysin. The scale bar represents 1 μm. D: HPLC chromatograms of thermolysin catalysed peptide synthesis in the course of time in the presence of 0.5 mg thermolysin. Peaks shown are normalised and represent Fmoc-Phe and Fmoc-(Phe)₃;

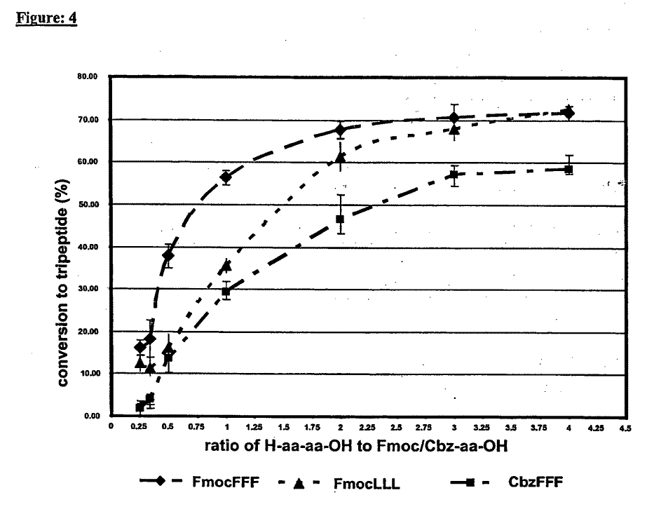

FIGS. 4-6 illustrate the results of Examples 2-4 respectively;

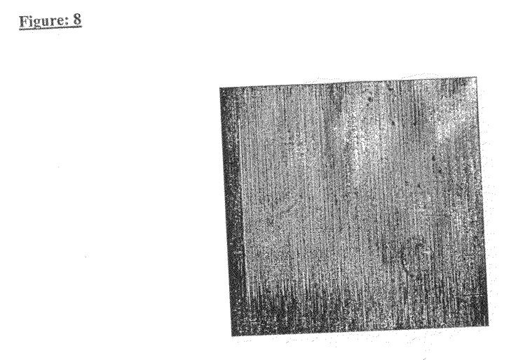

FIGS. 7 and 8 illustrate the results of Example 5; and

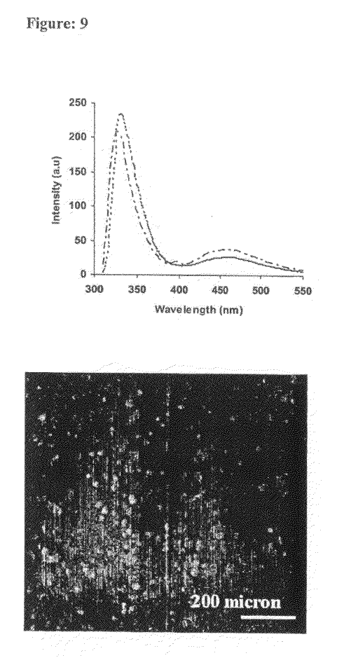

FIGS. 9-11 illustrate the results of Example 6-8 respectively.

### EXAMPLE 1

### Enzyme Triggered Self-Assembly of Peptide hydrogels Via Reversed Hydrolysis

The inventors conducted a series of experiments to investigate the use of the hydrolase enzyme, thermolysin, for synthesising peptides, and the subsequent preparation of hydrogel scaffolds.

### Materials and Methods

### (1) The Amino Acid and Peptide Precursors

A first objective was to determine combinations of amino acids and peptides that are capable of undergoing an enzyme triggered synthesis leading to the formation of Fmoc-protected peptides which could then self assemble into stable hydrogels. Suitable Fmoc-amino acids and peptide combinations would have to fulfill two requirements. Firstly, they should be recognized by the enzyme of choice and secondly, they should favour gel formation at physiological conditions.

The inventors started with a small library of Fmoc amino acids with different properties (as shown in FIG. 2B). The library was selected to cover a range of hydrophobicities (Gly (structure a in FIG. 2B), Ala (structure b), Val (structure c), Leu (structure e), Phe (structure f)). It was intended to use the hydrolase enzyme to synthesise a tripeptide, by reacting the Fmoc amino acids with Phe-Phe dipeptides or Leu-Leu dipeptides as will be described hereinafter.

### (2) The Hydrolase Enzyme

The enzyme that was selected for this study (thermolysin) is an endo-protease, which means that under physiological conditions, it would normally hydrolyse peptide bonds with more than one amino acid residue on either side of the cleaved amide bond. It was therefore decided to use two di-peptides (Phe-Phe, Leu-Leu) in combination with single Fmoc amino acids (as shown in FIG. 2B) that are known to be recognized by this enzyme.

The enzyme that was selected has complementary selectivities for the amino acids flanking the hydrolyzed (or in this case synthesized) amide bond (these amino acids are referred to as P1 and P′1). Thermolysin from thermoproteolyticus Rokko prefers hydrophobic residues (such as Phe-Phe and Leu-Leu) at the P′1 end of the cleaved peptide bond and is unselective for the P1 position (and should accept all Fmoc amino acids tested).

FIG. 2C gives a proposed self-assembly mechanism, that is directed by π-stacking of Fmoc-groups and may be further stabilized by hydrogen bonding.

In each case, equimolar amounts of 40 μmol of the Fmoc amino acid and dipeptide were mixed to give a suspension that was dissolved by addition of concentrated NaOH (0.5 M). This was followed by a gradual lowering of pH to a value of approximately 7 using a concentrated 0.1 M HCl solution. The final concentration was 12 mM (corresponding to <1% w/w). Upon addition of 2 mg enzyme thermolysin, the gelation started within minutes for each of the combinations of Fmoc-amino acids and peptides, as shown in Table 1.

TABLE 1
A number of Fmoc-amino acid/di-peptide combinations that
were tested in enzyme triggered hydrogel formation.

| | | | | % | |
|---|---|---|---|---|---|
| Entry | Fmoc-AA | Di-peptide | Enzyme | conversion | Gel formed? |
| 1 | Ala | Phe-Phe | thermolysin | 27 | ✓ |
| 2 | Val | Phe-Phe | thermolysin | 64 | ✓ |
| 3 | Leu | Phe-Phe | thermolysin | 51^{a} | ✓ |
| 4 | Phe | Phe-Phe | thermolysin | 45, 55^{b} | ✓ |
| 5 | Leu | Leu-Leu | thermolysin | 22^{a} | ✓ |

Conditions were 22° C. and pH 7, 40 μmol Fmoc amino acid and di-peptide, 2 mg enzyme powder in a total volume of 3.4 mL
^{a}mixture of Fmoc-peptides formed
^{b}60 μmol starting materials was used

To establish whether hydrogel formation represented formation of Fmoc-tripeptides HPLC analysis of the reaction mixtures was carried out. Successful enzyme triggered self-assembly of peptide hydrogels was observed in all cases, and in each case this observation correlated well with significant formation of Fmoc-peptides. In entries 3 and 5, mixtures of peptides were formed, including Fmoc-pentamers (formed by further coupling of di-peptides). It must be noted that equilibrium was not yet reached in all cases.

The surprising results demonstrate that the enzyme thermolysin can be used to selectively trigger peptide formation and hydrogel formation via a reversed hydrolysis process. The stability of the hydrogels varied from weaker gels to more stable self-supporting gels. The most stable hydrogel that was obtained was Fmoc-(Phe)₃ (entry 4), which was therefore investigated in more detail (as shown in FIG. 3). Here, a single Fmoc-tripeptide product was formed as demonstrated by mass spectrometry (sodium salt were detected at 704.3 Da). If the 45% conversion that was observed with 40 μmol represents the equilibrium position, it would be expected that an increase in hydrogel precursor concentration should lead to an increase in conversion. Indeed, when 60 μmol Fmoc-Phe and Phe-Phe were used, a conversion of 55% was obtained. For future applications where these hydrogel scaffolds may be used as scaffolds for 3D cell culture, the inventors believe that the gelation times should be as short as possible (in minutes) and that the gels are transparent (thus allowing for unimpaired optical interrogation of cells inside the gel matrix).

Referring to FIG. 3A, there is shown that a transparent hydrogel could be obtained within 5 minutes when 2 mg thermolysin was applied. Cryo-SEM analysis of the resulting gel revealed a microstructure of interwoven fibres of approx 10-20 nm in diameter (as shown in FIG. 3C). To investigate the minimal amount of enzyme required to trigger self-assembly, the enzyme amount was varied between 0.05 and 5 mg (as shown in FIGS. 3B and 3D). For amounts of enzyme of up to 2 mg an increase of the enzyme quantity resulted in a steady increase of the reaction rate up to 3.2 mmol/min/mg. When adding more enzyme, no further increase in the initial rate was observed, suggesting that the rate determining step is the self assembly. The critical enzyme amount used to obtain gelation within a reasonable amount of time was 0.5 mg. The amounts of enzyme used is still over that would be expected in natural contexts in vivo. The inventors believe that re-design and optimization of the peptide structures may increase the rates further.

### SUMMARY

In summary, the inventors have demonstrated for the first time that hydrolases (proteases) may be used to drive the synthesis and subsequent self-assembly of peptides to form a stable hydrogel under physiological conditions. While the inventors do not wish to be bound by any hypothesis, they believe that there are two factors that determine peptide self-assembly:—(i) whether the enzyme catalyses the reaction (specificity) and (ii) whether sufficient peptide is formed to start the overcome the critical concentration for self assembly to occur under the applied conditions. Thus, solutions of Fmoc amino acids and di-peptides transform into a nanofibrous hydrogel within minutes of addition of a protease. The inventors envisage that this method will may have implications for in situ gelation in tissue engineering. An estimated 2-3% of the mammalian proteome are proteases, and many of these are secreted by cells. Hence, there are opportunities to exploit these proteases to control gelation in vivo, under constant conditions of temperature, ionic strength, and pH (biologically acceptable conditions in man).

### EXAMPLE 2

This Example illustrates the ability of Fmoc- and Cbz (carboxybenzyl)-modified amino acids to react with dipeptides of the corresponding amino acids to form Fmoc- or Cbz-tripeptides. More specifically, the systems investigated were (i) Fmoc-F and FF, (ii) Fmoc-L and LL, (iii) Cbz-F and FF, and (iv) Fmoc-G- and GG.

### Reactant Preparation

Fmoc-amino acids or Cbz-amino acids with dipeptides were weighed in to a clean universal to the different concentrations and the various ratios required. They were then suspended in deionised water, and solubilized by the addition of 0.5M NaOH to adjust the pH to 13. The particle size was reduced through the reduction of larger clumps by gentle manipulation with a spatula, and the subsequent immersion in a sonication bath. This resulted in a homogenous, opaque suspension. The pH was reduced by the addition dropwise from a 200 μl pipette of 0.1 M HCl with gentle vortexing until the value reached 7. It was found that if the rate of HCl addition was increased, localised pH variations would lead to precipitation of the molecules, which, if excessive, were found to be irreversible. Upon acquisition of a neutral pH, de-ionised water was added up to the required volume.

### Enzyme Preparation

The enzyme, thermolysin, was provided in the form of a lyphophilized powder. This was weighed to a sterile universal, and then reconstituted in distilled water to a concentration of 1 mg/ml. In order to ensure the enzyme was fully solubilised, the solution was vortexed for thirty seconds, and stored at room temperature within a sealed container and used within thirty minutes.

### Enzymatic Reaction

The reactants formed were mixed 3:1 with the enzyme solution to the required volume in order to provide a final working concentration of 0.25 mg/ml. A self-supporting gel was observed to have occurred within twenty minutes, and as the reaction continued, the opaque solution was observed to have become clear over a longer time period.

### Optimisation

A 96 well format was developed to screen large numbers of combinations of reactant ratios and concentrations. The volume of each well was 300 ul. Relative amounts of Fmoc/Cbz-amino acids and dipeptide were added on ratios in a range from 0.25:1 to 4:1. Molarities tested were 20 mM (in terms of Fmoc/Cbz-aa). In order to enable this, 10 mM Fmoc/Cbz-aa and 160 mM di-peptide suspensions were produced. Relative quantities of these were added. A set volume of 25 ul of enzyme was added at a concentration of 12 mg/ml. Sufficient quantities of distilled water were added to make up to a final volume of 300 μm. As an example, to create a 20 mM 1:4 gel, 171.73 ul of 140 mM di-leucine, 75 ul of 80 mM Fmoc-Leucine, 25 ul of enzyme solution, and 28.57 ul of water were added to a well. This was then gently mixed with a pipette, and left to react for 48 hours at room temperature. Each reaction was performed in triplicate. Results of the reactions were measured by HPLC, and the degree of conversion of Fmoc/Cbz-amino acid to Fmoc/Cbz-tripeptide was calculated.

The results are shown in FIG. 4. FIG. 4 demonstrates. Fmoc-triGlycine (Fmoc-GGG) is produced as it doesn't form a gel. Fmoc-tri-phenylalanine (Fmoc-FFF) and Fmoc-tri-leucine (Fmoc-LLL) both gave yields of over 70% at a 1:4 ratio (i.e. Fmoc-a is at 20 mM, aa is at a concentration of 80 mM) whereas Cbz-Phe-Phe-Phe-OH gave a yield of 60%. Little Fmoc-triGlycline (Fmoc-GGG) was produced to the natureo f the thermodynamically controlled equilibrium.

### EXAMPLE 4

This Example demonstrates a “real time” study of the reaction of Fmoc-Phe-OH and Phe-Phe-OH in the presence of thermolysin.

The “Reactant Preparation” procedure described in Example 2 using 20 mM Fmoc-Phe and 80 mM of Phe-Phe. Additionally a solution of thermolysin was produced using the “Enzyme Preparation” procedure of Example 3.

The reactants formed were mixed with 3:1 with the enzyme solution to the required volume in order to provide a final working concentration of 0.25 mg/ml. The solution was mixed in a fluorescence cuvette and spectra were recorded at various time points on a Jasco FP-6500 with slit width of 2.5 nm, a medium speed scan from 275 to 550, and an excitation wavelength of 270 nm.

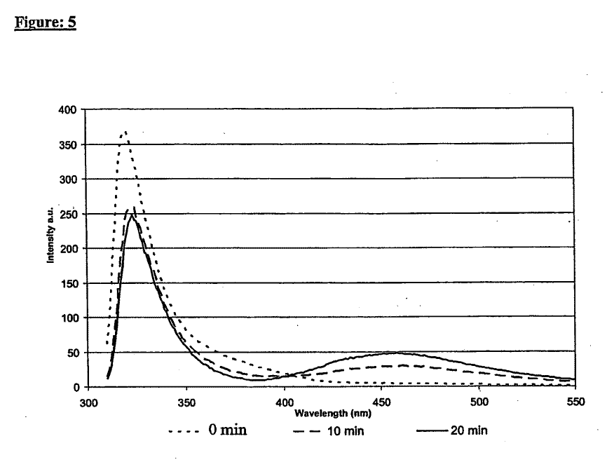

The results are shown in FIG. 5.

Referring to FIG. 5, the Fmoc monomer main peak at ˜320 nm and the excimer the shoulder at ˜350 nm. FIG. 5 demonstrates the quenching of the main peak A and the formation of the second peak, B with the gelation over time with the gel formed by the Fmoc-Phe-OH and Phe-Phe-OH with thermolysin. The trace shows evidence of the formation of a supramolecular aggregate which give rise to a peak at ˜450 nm.

The addition of enzyme leads to a red shift of the Fmoc signals. As can be seen, changes in relative peak height occurred upon gelation; the gel formed from solution, leading to a reduction in peak A. This reduction is likely due to quenching of the fluorophores as they form into fibers, and peak B increases with the formation of the supramolecular aggregate.

### EXAMPLE 5

Circular dichroism spectra over a time course were collected of Fmoc-Leu-OH (20 mM) and Leu-Leu-OH (80 mM) with thermolysin prepared following the procedure described in Example 4.

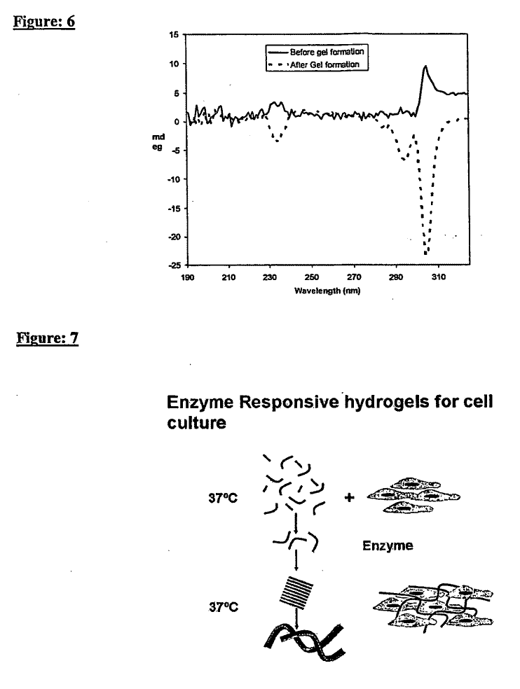

The circular dichroism spectra were measured on a Jasco J-810 spectrometer, using a 0.5 mm cuvette, 190-350 nm, with a 1 nm slit width and r second accumulation and 3 acquisitions. Due to absorbance at low wavelengths, it was only possible to monitor the Fmoc group. The results are shown in FIG. 6 which shows the circular dichroism spectra of a Fmoc-Leu-Oh+Leu-Leu-OH_thermolysin immediately after the addition of thermolysin and after 90 minutes. As demonstrated in FIG. 6, there is a clear change in the amplitude and direction of the Fmoc signal at 305 nm indicating a more ordered structure being formed.

### EXAMPLE 6

### In Situ Enzyme Triggered Hydrogel Assembly Application in 3D Cell Culture

The inventors then conducted a series of experiments to investigate the use of the hydrogel scaffolds formed by the hydrolase enzyme thermolysin as described in Example 1, in supporting tissue cell cultures.

Around 2-3% of the mammalian proteome are proteases, and many are secreted by cells. Examples include the matrix metalloproteases. Therefore, if the hydrogels could support a cell culture, then the inventors believe that such enzymes present in tissue fluids could be exploited to control gelation in situ, under constant conditions of temperature, ionic strength, and pH. Hence, the hydrogels could have significant uses in biological tissue regeneration and engineering, e.g. minimal invasive surgery as it would allow for in situ scaffold formation under constant conditions.

Referring to FIG. 7, there is shown a schematic showing how an enzyme found in vivo could be exploited to cause gelation at body temperature, i.e. 37° C. The inventors used the enzyme thermolysin, as discussed in Example 1 to trigger gelation of the hydrogel in the following experiments.

### 1) Thermolysin-Triggered Hydrogels

The following results were obtained for the thermolysin-triggered hydrogels in the presence of 1 ml of bovine chondrocyte cells. In this experiment, cells were added after enzymatic gel formation.

### Materials and Methods

Bovine chondrocytes were isolated from cartilage of the proximal side of the metacarpalphalangeal joint, washed in PBS and incubated overnight in Dulbeccos Modified Eagles Medium (DMEM) supplemented with 10% foetal calf serum, 100 units ml⁻¹ penicillin/streptomycin and 0.85 mM ascorbic acid. The cartilage was finely chopped and incubated with pronase type E (700 units ml⁻¹′) (BDH Ltd., Poole, UK) in medium for 2 h followed by collagenase type 1a (300 units ml⁻¹) (Sigma-Aldrich Co. Ltd., Poole, UK) in medium for 2 h. The cell suspension was centrifuged at 1500 rpm for 5 min to pellet the cells. The cells were washed twice in medium and seeded directly onto the peptide scaffold precursors and varying amount of enzyme (0.1-2 mg) enzyme was added. The cultures were maintained in an incubator at 37° C. with a humidified atmosphere of 5% CO₂ for up to 4 weeks. Chondrocytes were used up to passage 5.

The results obtained for already made hydrogels with 0.5 mg of enzyme are shown in Table 2.

TABLE 2
Results obtained after one week of cell culture (1 ml of
cells/media) for different Fmoc-Phe-Phe-Phe-OH samples

| | Sample aspect after | |
|---|---|---|
| | one week incubation | Presence of |
| Sample description | at 37° C. | cells |
| Fmoc-Phe-OH + H-Phe-Phe-OH (40 | The “gel” | ✓ |
| μmol) with 0.5 mg of thermolysin, | structure was | |
| one week after the formation of | retained | |
| the hydrogel | | |

Light microscopy was then carried out to analyse the hydrogel for the presence of cells. As shown in FIG. 8, there is shown the presence of cells revealed by light microscopy of hematoxylin-stained Fmoc-Phe-Phe-Phe-OH hydrogel. The presence of cells is revealed after one week of incubation at 37° C. The light microscope images were taken at different depths of the samples.

Hence, FIG. 8 shows cells stained with hematoxylin, and illustrates that the cells are not only present at the surface of the hydrogel, but are incorporated in the hydrogel structure.

### SUMMARY

The data show that thermolysin is a powerful enzyme in hydrogel synthesis, producing strong and stable hydrogels. Furthermore, the hydrogels made by thermolysin are able to support cell cultures. The inventors believe therefore that hydrolase enzymes in the body may be harnessed to synthesis peptides which can self-assemble to form a hydrogel. The hydrogel can then act as a cell scaffold for tissue repair or regeneration.

### EXAMPLE 7

This Example demonstrates the advantages of the approach employed in the present invention as compared to the application of other specific stimuli for hydrogel formation.

The formation of supramolecular assemblies is usually initiated by the application of specific stimuli such as a change in temperature (typically cooling), diluting from organic solvents, using the addition of salts to alter ionic strength, or the use of a pH change as a trigger. However, a major disadvantage of these methods is that “ensuring that the components aggregate in a specific motif remains a formidable task; molecular components are easily entrapped in kinetically stable arrangements of varying topology.” (See Jonkheijm, P., Van der Schoot, P., Schenning, A. P. H. J., Meijer, E. W., Probing Solvent-Assisted Nucleation Pathway in Chemical Self-Assembly, Science, 2006, 313, 80-83). A few examples exist of the use of enzymatic methods using a kinetically controlled approach (for example driven by ATP coupled phosphorylations). The difficulty with these methods that it is a major challenge to perform the reaction very close to thermodynamic equilibrium. Enzymes have also been used for hydrolytic cleavage of disrupting groups to form hydrogelators from non-gelling precursors. Our invention describes an enzyme triggered method that acts under thermodynamic control and releases a molecule of water for each hydrogelator molecule that is formed. Using a thermodynamically controlled method kinetically trapped states are avoided, leading to the formation of high quality homogeneous assemblies with fewer assembly defects.

The advantage of the present invention is demonstrated with reference to FIG. 9. The left hand side of FIG. 9 shows a comparison of the fluorescence emission (excitation 270 nm) of acid/base triggered gelation (dotted) and enzyme triggered gelation (dot dash), the peak at 450 nm increased indicating increased order in enzyme triggered experiment. The right hand image shows live/dead staining of cells in acid-base triggered matrix (left) and enzyme triggered gelation (right). The acid-base triggered gels are inhomogeneous and cell behaviour is patchy resulting in regions of dead cells, while the enzyme triggered gelation is more homogeneous with mainly live cells.

### EXAMPLE 8

This Example demonstrates the effect of enzyme-triggered Fmoc-FFF and Fmoc-LLL gel systems on human mesenchymal stem cell (MSC) viability and morphology was investigated.

Bone marrow was obtained, with patient and ethical consent, from patients undergoing hip replacement surgery. Human MSCs were isolated using a histopaque gradient and cultured for 5 days in α-MEM containing 10% heat-inactivated foetal calf serum, 100 U/ml streptomycin/penicillin and 0.85 mM ascorbic acid. At this point non-adherent cells were removed using media washes and adherent MSCs were cultured to 80% confluence in passage 3. Cells were then trypsinised, counted and an appropriate number centrifuged.

The cell pellets were then resuspended in either enzyme-triggered Fmoc-FFF or Fmoc-LLL solutions to a density of 4×10⁶/ml. For every 1 ml final volume 750 μl of gel solution was used and supplemented with 250 μl of a 1 mg/ml thermolysin (Fluka) enzyme solution in Hanks Buffered Saline Solution (HBSS; Invitrogen).

The solutions were mixed thoroughly and 200 μl aliquots were pipetted into high pore density (0.4 μm pore size) cell culture inserts in 24-well plates. Gels were allowed to polymerise and then media (α-MEM as previously described) was added gently to both the well and the insert. The cell-seeded gels were then cultured under standard conditions for 1 and 7 days with media changed after 2, 4 and 6 days.

Following the culture periods trypan blue exclusion assays were performed by adding 20 μl of trypan blue to each gel layer and pipetting to mix. A 10 μl aliquot was then transferred to a microscope slide, a coverslip placed on top and cell morphology and viability examined using an inverted light microscope. This demonstrated the presence of viable cells in both gel systems following both 1 and 7 days in culture. Furthermore these cells retained a rounded morphology throughout the time course of the experiment.

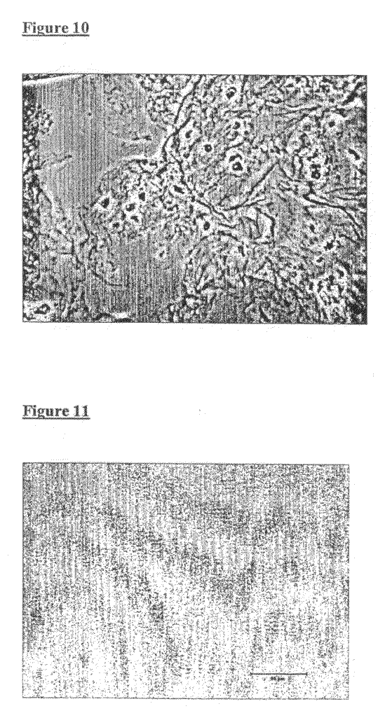

FIG. 10 illustrates live MSC cells in Fmoc-LLL gels.

### EXAMPLE 9

This Example demonstrates culture of bovine chondrocytes.

Bovine chondrocytes were isolated from cartilage of the proximal side of the metacarpalphalangeal joint, washed in PBS and incubated overnight in Dulbeccos Modified Eagles Medium (DMEM) supplemented with 10% foetal calf serum, 100 units ml⁻¹ penicillin/streptomycin and 0.85 mM ascorbic acid.

The cartilage was finely chopped and incubated with pronase type E (700 units mL⁻¹) (BDH Ltd., Poole, UK) in medium for 2 h followed by collagenase type 1a (300 units mL⁻¹) (Sigma-Aldrich Co. Ltd., Poole, UK) in medium for 2 h.

The cell suspension was centrifuged at 1500 rpm for 5 min to pellet the cells. The cells were washed twice in medium and seeded directly into the peptide solution at a cell density of 5×10⁵ ml⁻¹ in medium.

900 μl of Fmoc-Leu-OH and Leu-Leu-OH solution was placed in a 24 well plate. 200 μl of chrondocyte cell suspension and 100 μL of enzyme (thermolysin) was then added to the plate, mixed together and left it to get settled for a few minutes. This was then placed in the incubator at 37° C. with a humidified atmosphere of 5% CO₂. A microscopic image was taken after 24 hours of incubation and is shown in FIG. 11.

1. A method of preparing a hydrogel, the method comprising:
(i) reacting
a first molecule comprising a carboxylic acid group; and
a second molecule comprising an amine group or an alcohol group;
with a hydrolase enzyme to form a product comprising an amide bond or ester bond,
wherein under normal physiological conditions the hydrolase enzyme catalyzes the production of an amine or an alcohol from an amide or an ester; and
(ii) maintaining the product comprising an amide or ester bond under conditions suitable to allow hydrogel formation.

2. The method of claim 1, wherein the second molecule comprises an amine group and the hydrolase is a protease.

3. The method of claim 2, wherein both the first and second molecules are selected from the group consisting of amino acids, peptides and derivatives thereof.

4. The method of claim 3, wherein at least one of the first and second molecules comprises a phenylalanine residue.

5. The method of claim 3, wherein at least one of the first and second molecules comprises a leucine residue.

6. The method of claim 3, wherein at least one of the first and second molecules comprises an isoleucine residue.

7. The method of claim 1, wherein the second molecule comprises an alcohol group and the hydrolase is an esterase.

8. The method of claim 7, wherein at least one of the first and second molecules comprises a fatty acid.

9. The method of claim 1, wherein at least one of the first and second molecules further comprises an aromatic stacking ligand.

10. The method of claim 9, wherein the aromatic stacking group is Fmoc (fluorenylmethoxycarbonyl).

11. A hydrogel prepared according to the method defined by claim 1.

12. A pharmaceutical composition comprising the hydrogel of claim 11 and a pharmaceutically acceptable vehicle.

13. A method of treating tissue loss/damage, comprising administering a therapeutically effective amount of a hydrogel of claim 11 to a subject suffering from a medical condition characterized by tissue loss/damage.

14. The method of claim 13, wherein the tissue loss/damage is caused by a wound or related injury.

15. The method of claim 14, wherein the wound or related injury is selected from the group consisting of a chronic wound, an abrasive wound, a wound formed by pressure, an acute penetrative wound and a wound arising as a result of a crush to the body.

16. The method of claim 13, wherein the tissue loss/damage is caused by a tissue degenerative disorder.

17. The method of claim 16, wherein the tissue degenerative disorder is selected from the group consisting of a neurodegenerative disorder, an intervertebral disc disorder, cartilage degeneration, bone degeneration, osteoporosis, a liver degenerative disorder, a kidney degenerative disorder, and muscle atrophy.

18. A pharmaceutical composition comprising:
a first molecule comprising a carboxylic acid group;
a second molecule comprising an amine group or an alcohol group; and
a hydrolase enzyme,
wherein at least one of the first or second molecules comprises an aromatic stacking group.

19. A method of treating tissue loss/damage, comprising administering to a subject suffering from a medical condition characterized by tissue loss/damage an effective amount of a composition of claim 18.

20. A cell culture medium comprising a hydrogel of claim 11, and at least one cell.

21. A method of wound repair or treatment of a tissue degenerative disorder, comprising forming a cell-supporting hydrogel comprising a hydrogel and at least one cell, wherein said forming is conducted (a) in situ in a body area to be treated, or (b) in vitro followed by transferring said cell-supporting hydrogel to the area.
